(19) United States
(12) Patent Application Publication
(10) Pub. No.: US 2014/0205569 A1
(43) Pub. Date: Jul. 24, 2014

(54) METHODS AND COMPOSITIONS FOR PREVENTING AND TREATING OSTEOARTHRITIS

(71) Applicant: Nierissa NIELSEN, Kingsburg, CA (US)

(72) Inventors: Rondo Paul Middleton, Creve Coeur, MO (US); Mark Kenneth Waldron, St. Louis, MO (US); Nerissa Nielsen, Kingsburg, CA (US)

(21) Appl. No.: 13/996,225

(22) PCT Filed: Dec. 15, 2011
(86) PCT No.: PCT/US11/65173

§ 371 Date: Dec. 9, 2013

Related U.S. Application Data
(60) Provisional application No. 61/459,902, filed on Dec. 21, 2010.

(51) Int. Cl. A61K 31/202; A23L 1/30; A61K 45/06

(52) U.S. Cl. CPC A61K 31/202; A61K 45/06; A23L 1/3008
USPC 424/93.1; 514/560; 554/224

(57) ABSTRACT

The invention provides methods for preventing and treating osteoarthritis, preventing and treating the degradation of articular cartilage, and promoting and maintaining joint health in an animal. The methods comprise administering DGLA to the animals, preferably in amounts of from about 0.01 to about 100 mg/kg/day.

### CROSS REFERENCE TO RELATED APPLICATIONS IONS

This application claims priority to U.S. Provisional Application Ser. No. 61/459902 filed Dec. 21, 2010, the disclosure of which is incorporated herein by this reference.

### BACKGROUND OF THE INVENTION

1. Field of the Invention

The invention relates generally to methods for preventing and treating osteoarthritis and particularly to methods for using dihomo-γ-linolenic acid (DGLA) for preventing and treating osteoarthritis.

2. Description of Related Art

Osteoarthritis (OA), also commonly referred to as degenerative joint disease, is characterized by the imbalance between the synthesis and degradation of articular cartilage. The breakdown of joint cartilage often results in joint pain and loss of mobility. OA commonly affects the hands, feet, spine, and the large weight bearing joints, such as the hips and knees. Symptoms may include joint pain, tenderness, stiffness, locking, and sometimes an effusion. A variety of causes, such as hereditary, developmental, metabolic, and mechanical, may initiate processes leading to loss of cartilage.

DGLA is a 20-carbon 3 double bond ω-6 fatty acid (20:3n-6). DGLA can be synthesized directly from γ-linolenic acid (GLA) via chain elongation. In the body, DGLA only appears as an intermediate in biosynthesis and does not accumulate in any appreciable concentrations. However, when DGLA is given as a supplement concentrations capable of demonstrating therapeutic benefit are achieved. DGLA competes with arachidonic acid (AA) for cyclooxygenase (COX) and lipoxygenase enzymes, inhibiting the proinflammatory eicosanoids derived from AA. U.S. Pat. No. 5,763,484 discloses a method of treatment of a disorder, including breast cancer and other cancer, with effective amounts of one or more of the metabolites of lineolic acid, including dihomo-γ-linolenic acid, and one or more of the metabolites of α-lineolic acid. U.S. Pat. No. 6,177,470 discloses methods of treatment using an ascorbic-6-acid ester of γ-linolenic acid or an ascorbic-6-acid ester of dihomo-γ-linolenic acid for various conditions. U.S. Patent Application No. 20080108699 discloses compositions comprising dihomo-γ-linolenic acid as an active ingredient for the treatment of various skin diseases.

Osteoarthritis is a painful condition and is adverse to the health and wellness of an animal and result in a lower quality of life for an animal. There is, therefore, a need for methods and compositions useful for promoting the health and wellness and improving the quality of life for animals by preventing and treating osteoarthritis.

### SUMMARY OF THE INVENTION

It is, therefore, an object of the invention to provide methods and compositions useful for preventing and treating osteoarthritis, preventing and treating the degradation of articular cartilage, and promoting and maintaining joint health.

It is another object of the invention to provide methods for promoting the health and wellness of animals.

It is another object of the invention to provide methods for extending the prime years of an animal's life.

One or more of these or other objects are achieved by administering DGLA to animals in therapeutically effective amounts for preventing and treating osteoarthritis, preventing and treating the degradation of articular cartilage, and promoting and maintaining joint health. In general embodiments, DGLA is administered to the animals in amounts of from about 0.01 to about 100 milligrams per kilogram of body weight per day (g/kg/day) for as long as there is a need for such treatment.

Other and further objects, features, and advantages of the invention will be readily apparent to those skilled in the art.

### DETAILED DESCRIPTION OF THE INVENTION

### Definitions

The term “animal” means any animal that has a need for preventing or treating osteoarthritis, preventing or treating the degradation of articular cartilage, and promoting and maintaining joint health in an animal, including human, avian, bovine, canine, equine, feline, hicrine, lupine, murine, ovine, or porcine animals.

The term “companion animal” means domesticated animals such as cats, dogs, rabbits, guinea pigs, ferrets, hamsters, mice, gerbils, horses, cows, goats, sheep, donkeys, pigs, and the like.

The term “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “treating”, “treat”, and “treatment” embrace both preventative, i.e., prophylactic, and palliative treatment.

The terms “pharmaceutically acceptable” and “nutraceutically acceptable” indicates that the substance or composition must be compatible chemically and/or toxicologically, with the other ingredients comprising a formulation, and/or the mammal being treated therewith.

The term “health and/or wellness of an animal” means the complete physical, mental, and social well being of the animal, not merely the absence of disease or infirmity.

The term “extending the prime” means extending the number of years an animal lives a healthy life and not just extending the number of years an animal lives, e.g., an animal would be healthy in the prime of its life for a relatively longer time.

The term “degenerative joint disorder” refers to any etiological or pathological symptom affecting a joint. Such symptoms and etiology include swelling, pain and/or stiffness of a joint such that complete or partial loss of function and/or damage to the joint and/or a reduction of the joint mobility. The term also includes chronic inflammation, primarily of the synovial tissue, pannus formation, destruction of articular cartilage and release of various enzymes, for example, collagenase and lysosomal enzymes, in an affected joint, osteoarthritis, rheumatic disorders with cartilage breakdown, rheumatoid arthritis, chondrolysis after joint trauma, for example, after meniscus or patella injuries or torn ligaments, or chondrolysis associated with prolonged immobilization of joints. By way of example, degenerative joint disorder includes osteoarthritis, rheumatoid arthritis, juvenile rheumatoid arthritis, psoriatic arthritis, tendonitis, ankylosing spondilitis, bursitis, spinal disc injuries, and temporomandibular joint disorder.

The term “unsaturated fatty acids” or “UFA” means polyunsaturated fatty acids or monounsaturated fatty acids, including monocarboxylic acids having at least one double bond. UFAs include omega-6 (ω-6) fatty acids such as linoleic acid (LA) and arachidonic acid (AA) and omega-3 (ω-3) fatty acids such as eicosapentaenoic acid (EPA), alpha-linolenic acid (ALA), docosapentaenoic acid (DPA) and docosahexaenoic acid (DHA). UFAs also include myristoleic acid, palmitoleic acid, oleic acid, cis-vaccenic acid, and erucic acid.

The term “fish oil” means a fatty or oily extract, relatively rich in UFA, whether crude or purified, obtained from a sea animal, preferably a cold-water fish such as, but not limited to, salmon, tuna, mackerel, herring, sea bass, striped bass, halibut, catfish, and sardines, as well as shark, shrimp, and clams, or any combination thereof. Fish oil is generally a term of art used by ingredient suppliers and encompasses a range of products of varying UFA content and purity.

The term “in conjunction” means that DGLA or other compounds or other compositions of the invention are administered to an animal (1) together in a food composition or (2) separately at the same or different frequency using the same or different administration routes at about the same time or periodically. “Periodically” means that DGLA or other compounds or other compositions are administered on a schedule acceptable for specific compounds or compositions. “About the same time” generally means that DGLA or other compounds or compositions are administered at the same time or within about 72 hours of each other.

The term “dietary supplement” means a product that is intended to be ingested in addition to a normal animal diet. Dietary supplements may be in any form, e.g., solid, liquid, gel, tablet, capsule, powder, and the like. Preferably they are provided in convenient dosage forms, e.g., in sachets. Dietary supplements can be provided in bulk consumer packages such as bulk powders, liquids, gels, or oils. Similarly such supplements can be provided in bulk quantities to be included in other food items such as snacks, treats, supplement bars, beverages, and the like.

The term “aging” means being of an advanced age such that an animal has reached or exceeded 50% of the average life expectancy for the animal's species and/or breed within such species. For example, if the average life expectancy for a given breed of dog is 12 years, then an “aging animal” within that breed is 6 years old or older.

The term “food” or “food product” or “food composition” means a product or composition that is intended for ingestion by an animal, including a human, and provides nutrition to the animal.

The term “regular basis” means at least monthly dosing with DGLA and more preferably weekly dosing. More frequent dosing or consumption, such as twice or three times weekly, is preferred in certain embodiments. Still more preferred are regimens that comprise at least once daily consumption, e.g., when DGLA is a component of a food composition that is consumed at least once daily.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of individual DGLA and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The dosages expressed herein are in milligrams per kilogram of body weight per day (mg/kg/day) unless expressed otherwise.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for preventing and treating osteoarthritis in an animal. The methods comprise administering to the animal a therapeutically effective amount of DGLA.

In another aspect, the invention provides methods for preventing and treating the degradation of articular cartilage in an animal. The methods comprise administering to the animal a therapeutically effective amount of DGLA.

In another aspect, the invention provides methods for promoting and maintaining joint health in an animal. The methods comprise administering to an animal a therapeutically effective amount of DGLA.

In a further aspect, the invention provides methods tor promoting the health and wellness of an animal. The methods comprise administering to an animal a therapeutically effective amount of DGLA.

In another aspect, the invention provides methods for extending the prime years of an animal's life. The methods comprise administering DGLA to an animal therapeutically effective amount of DGLA.

The inventions are based upon the discovery that DGLA is able to positively modulate osteoarthritis markers. Specifically, DGLA is able to inhibit NF-kB activation and reduce inflammatory mediators. Genes associated with osteoarthritis have been previously identified in WO2005075685. The methods of the invention may also be useful in the treatment of inflammatory related conditions, cancer metastasis and other degenerative joint disorders.

In the methods of the invention, DGLA is administered to an animal in amounts of from about 0.01 to about 100 mg/kg/day, preferably from about 0.05 to about 50 mg/kg/day, most preferably from about 0.1 to about 10 mg/kg/day. In various embodiments, the DGLA is administered in amounts of from about 0.5 to about 5 mg/kg/day.

DGLA can be derived from any suitable source. There are very few natural sources of DGLA. Due to the recent progress in fermentation technology, DGLA can be derived from organisms such as microalgae, fungi, bacteria and yeast, In a preferred embodiment DGLA is derived from microalgae, more preferably from the IKG-1 microalgae strain. In another embodiment, DGLA is derived from fungi, preferably fungi belonging to the order Mucorales. Examples of fungi belonging to the order Mucorales include but are not limited to Mortierella, Pythium and Entomophyhora. In a preferred embodiment, DGLA is derived from Mortierella, more preferably Mortierella alpinia.

DGLA can be administered to the animal in any suitable form using any suitable administration route. For example, DGLA can be administered in a DGLA composition, in a food composition, in a dietary supplement, in a pharmaceutical composition, in a nutraceutical composition, or as a medicament. Similarly, DGLA can be administered using a variety of administration routes, including oral, intranasal, intravenous, intramuscular, intragastric, transpyloric, subcutaneous, rectal, and the like. Preferably, DGLA is administered to an animal orally. Most preferably, DGLA is administered orally to an animal as a dietary supplement or as an ingredient in a food composition.

In a preferred embodiment, DGLA is administered to an animal as an ingredient in a food composition suitable for consumption by an animal, including humans and companion animals such as dogs and cats. Such compositions include complete foods intended to supply the necessary dietary requirements for an animal or food supplements such as animal treats.

In various embodiments, food compositions such as pet food compositions or pet treat compositions comprise from about 5% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include beef, pork, lamb, equine, poultry, fish, and mixtures thereof.

The food compositions may further comprise from about 5% to about 40% fat. Examples of suitable fats include animal fats and vegetable fats. Preferably the fat source is an animal fat source such as tallow or poultry fat. Vegetable oils such as corn oil, sunflower oil, safflower oil, grape seed oil, soy bean oil, olive oil and other oils rich in monounsaturated and polyunsaturated fatty acids, may also be used.

The food compositions may further comprise from about 10% to about 60% carbohydrate. Examples of suitable carbohydrates include grains or cereals such as rice, corn, millet, sorghum, alfalfa, barley, soybeans, canola, oats, wheat, rye, triticale and mixtures thereof. The compositions may also optionally comprise other materials such as dried whey and other dairy by-products.

The moisture content for such food compositions varies depending on the nature of the food composition. The food compositions may be dry compositions (e.g., kibble), semi-moist compositions, wet compositions, or any mixture thereof. In a preferred embodiment, the composition is a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one preferred embodiment, the compositions have moisture content from about 5% to about 20%. Dry food products include a variety of foods of various moisture contents, such that they are relatively shelf-stable and resistant to microbial or fungal deterioration or contamination. Also preferred are dry food compositions that are extruded food products such as pet foods or snack foods for either humans or companion animals.

The food compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those or ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the invention to the immune system of an animal.

In some embodiments, the ash content of the food composition ranges from less than 1% to about 15%, preferably from about 5% to about 10%.

In a preferred embodiment, the composition is a food composition comprising DGLA and from about 15% to about 50% protein, from about 5% to about 40% fat, from about 5% to about 10% ash content, and having a moisture content of about 5% to about 20%. In other embodiments, the food composition further comprises prebiotics or probiotics as described herein.

When administered in a food composition, DGLA comprises from about 0.001 to about 40% of the food composition, preferably from about 0.0025 to about 30%. more preferably from about 0.005 to about 20%. In various embodiments, food compositions comprise about 1%, 2%, 4%, 6%, 8%, 10%, 12%, 14%, 16%, 18%, 20%, 22%, 24%, 26%. 28%, 30%, 32%, 34%. 36%, 38%, or 40%.

In another embodiment, DGLA is administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise DGLA and optional compounds such as vitamins, preservatives, probiotics, prebiotics, and antioxidants. This permits the supplement to be administered to the animal in small amounts, or in the alternative can be diluted before administration to an animal. The dietary supplement may require admixing with a food composition or with water or other diluent prior to administration to the animal. When administered in a dietary supplement, DGLA comprises front about 0.001 to about 90% of the supplement, preferably from about 0.0025 to about 70%, more preferably from about 0.005 to about 60%.

In another embodiment, DGLA is administered to an animal in a pharmaceutical or nutraceutical composition. The pharmaceutical composition comprises DGLA and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, DGLA comprises from about 0.001 to about 90% of the composition, preferably from about 0.0025 to about 70%, more preferably front about 0.005 to about 60%.

DGLA can be administered to the animal on an as-needed, on an as-desired basis, or on a regular basis. A goal of administration on a regular basis is to provide the animal with a regular and consistent dose of DGLA or the direct or indirect metabolites that result from such ingestion. Such regular and consistent dosing will tend to create constant blood levels of DGLA and its direct or indirect metabolites. Thus, administration on a regular basis can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, DGLA may be administered directly to the animal, e.g., orally or otherwise. DGLA can alternatively be contacted with, or admixed with, daily feed or food, including a fluid, such as drinking water, or an intravenous connection for an animal that is receiving such treatment. Administration can also be carried out as part of a dietary regimen for an animal. For example, a dietary regimen may comprise causing the regular ingestion by the animal of DGLA in an amount effective to accomplish the methods of the invention.

According to the methods of the invention, DGLA administration, including administration as part of a dietary regimen, can span a period ranging from parturition through the adult life of the animal. In various embodiments, the animal is a human or companion animal such as a dog or cat. In certain embodiments, the animal is a young or growing animal. In more preferred embodiments, the animal is an aging animal. In other embodiments administration begins, for example, on a regular or extended regular basis, when the animal has reached more than about 30%, 40%, or 50% of its projected or anticipated lifespan. In some embodiments, the animal has attained 40, 45, or 50% of its anticipated lifespan. In yet other embodiments, the animal is older having reached 60, 66, 70. 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

DGLA is administered to an animal for a time required to accomplish one or more objectives of the invention, e.g., preventing and treating osteoarthritis in an animal; preventing and treating the degradation of articular cartilage; promoting and maintaining joint health; improving the quality of life; and promoting the health and wellness in an animal. Preferably, DGLA is administered to an animal on a regular basis.

In another aspect, the invention provides compositions comprising DGLA in a therapeutically effective amount for one or more of preventing and treating osteoarthritis in an animal; preventing and treating the degradation of articular cartilage in an animal; promoting and maintaining joint health in an animal; improving the quality of life in an animal; and promoting the health and wellness in an animal. The compositions contain DGLA in amounts sufficient to administer DGLA to an animal in amounts of from about 0.01 to about 100 mg/kg/day, preferably from about 0.01 to about 50 mg/kg/day, most preferably from about 0.05 to about 10 mg/kg/day when the compositions are administered as anticipated or recommended for a particular composition. Typically, DGLA comprises from about 0.001 to about 90% of a composition, preferably from about 0.005 to about 70%, more preferably from about 0.01 to about 60%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, 40%, 45%, 50%, 55%, 60%, 70%, or 80%,

DGLA compositions such as food, dietary, pharmaceutical, and other compositions may further comprise one or more substances such as vitamins, minerals, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat soluble vitamins as A, D, E, and K. Inulin, amino acids, enzymes, coenzymes, and the like may be useful to include in various embodiments.

In various embodiments, the DGLA compositions contain at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the DGLA compositions (e.g., uniformly or non-uniformly distributed in the compositions) or applied to the DGLA compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain is selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacteium sp. NCC2657 or Enterococcus faecium SF68 (NCIMB 10415). The DGLA compositions contain probiotics in amounts sufficient to supply from about 10⁴ to about 10¹² cfu/animal/day, preferably from 10⁵ to about 10¹¹ cfu/animal/day, most preferably from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP 1482811B1, EP1296565B1, and U.S. Pat. No. 6,929,793. In a preferred embodiment, the probiotic is Enterococcus faecium SF68 (NCINB 10415). In one embodiment, the probiotics are encapsulated in a carrier using methods and materials known to skilled artisans.

As stated, the DGLA compositions may contain one or more prebiotics, e.g., fructo-oligosaccharides, aluco-oligosaccharides, galacto-oligosaccharides, isomalto-oligosaccharides, xylo-oligosaccharides, soybean oligosaccharides, lactosucrose, lactulose, and isomaltulose. In one embodiment, the prebiotic is chicory root, chicory root extract, inulin, or combinations thereof. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, preferably from about 0.3 to about 7%, most preferably from about 0.5 to 5%, on a day matter basis. The prebiotics can be integrated into the compositions using methods known to skilled artisans, e.g., U.S. Pat. No. 5,952,033.

Compositions and methods of the invention may be used in combination with other unsaturated fatty acids. In some embodiments, the other unsaturated fatty acids include but are not limited to eicosapentaenoic acid, alpha-linolenic acid, docosapentaenoic acid, and docosahexaenoic acid. In a preferred embodiment, the other unsaturated fatty acid is eicosapentaenoic acid.

A skilled artisan can determine the appropriate amount of DGLA, food ingredients, vitamins, minerals, probiotics, prebiotics, antioxidants, or other ingredients to be use to make a particular composition to be administered to a particular animal. Such artisan can consider the animal's species, age, size, weight, health, and the like in determining how best to formulate a particular composition comprising DGLA and other ingredients. Other factors that may be considered include the type of composition (e.g., pet food composition versus dietary supplement), the desired dosage of each component, the average consumption of specific types of compositions by different animals (e.g., based on species, body weight, activity/energy demands, and the like), and the manufacturing requirements for the composition.

In a further aspect, the invention provides kits suitable for administering DGLA to animals. The kits comprise in separate containers in a single package or in separate containers in a virtual package, as appropriate for the kit component, DGLA and one or more of (1) one or more ingredients suitable for consumption by an animal; (2) instructions for how to combine DGLA and other kit components to produce a composition useful for preventing and treating osteoarthritis, preventing and treating the degradation of articular cartilage, promoting and maintaining joint health; (3) instructions for how to use DGLA for preventing and treating osteoarthritis; (4) instructions for how to use DGLA for preventing and treating the degradation of articular cartilage; (5) instructions for how to use DGLA for promoting and maintaining joint health; (6) one or more probiotics; (7) one or more inactivated probiotics; (8) one or more components of inactivated probiotics that promote health benefits similar to or the same as the probiotics, e.g., proteins, lipids, glycoproteins, and the like; (9) one or more prebiotics; (10) a device for preparing or combining the kit components to produce a composition suitable for administration to an animal; and (11) a device for administering the combined or prepared kit components to an animal. In one embodiment, the kit comprises DGLA and one or more ingredients suitable for consumption by an animal. In another embodiment, the kit comprises instructions for how to combine DGLA and the ingredients to produce a composition useful for preventing and treating osteoarthritis, preventing and treating the degradation of articular cartilage, and promoting and maintaining joint health.

When the kit comprises a virtual package, the kit is limited to instructions in a virtual environment in combination with one or more physical kit components. The kit contains DGLA and other components in amounts sufficient for preventing and treating osteoarthritis, preventing and treating the degradation of articular cartilage, and promoting and maintaining joint health. Typically, DGLA and the other suitable kit components are admixed just prior to consumption by an animal. The kits may contain the, kit components in any of various combinations and/or mixtures. In one embodiment, the kit contains a packet containing DGLA and a container of food for consumption by an animal. The kit may contain additional items such as a device for mixing DGLA and ingredients or a device for containing the admixture, e.g., a food bowl. In another embodiment, DGLA is mixed with additional nutritional supplements such as vitamins and minerals that promote good health in an animal. The components are each provided in separate containers in a single package or in mixtures of various components in different packages. In preferred embodiments, the kits comprise DGLA and one or more other ingredients suitable for consumption by an animal. Preferably such kits comprise instructions describing how to combine DGLA with the other ingredients to form a food composition for consumption by the animal, generally by mixing DGLA with the other ingredients or by applying DGLA to the other ingredients, e.g., by sprinkling DGLA on a food composition.

In a further aspect, the invention provides a means for communicating information about or instructions for one or more of (1) using DGLA for preventing and treating osteoarthritis; (2) using DGLA for preventing and treating degradation of articular cartilage; (3); using DGLA for promoting and maintaining joint health; (4) contact information for consumers to use if they have a question regarding the methods and compositions of the invention; and (5) nutritional information about DGLA. The communication means is useful for instructing on the benefits of using the invention and communicating the approved methods for administering DGLA and food compositions containing DGLA to an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising DGLA and one or more other ingredients suitable for consumption by an animal, e.g., one or more of protein, fat, carbohydrate, fiber, vitamins, minerals, probiotics, prebiotics, and the like. The methods comprise admixing one or more ingredients suitable for consumption by an animal with DGLA. Alternatively, the methods comprise applying DGLA alone or in conjunction or combination with other ingredients onto the food composition, e.g., as a coating or topping. DGLA can be added at any time during the manufacture and/or processing of the food composition. The composition can be made according to any method suitable in the art.

In another aspect, the invention provides a package useful for containing compositions of the invention. The package comprises at least one material suitable for containing DGLA and a label affixed to the package containing a word or words, picture, design, acronym, slogan, phrase, or other device, or combination thereof that indicates that the contents of the package contains DGLA. In some embodiments, the label affixed to the package contains a word or words, picture, design, acronym, slogan, phrase, or other device, or combination thereof that indicates that the contents of the package contains DGLA with beneficial properties relating to osteoarthritis. Typically, such device comprises the words “prevents and treats osteoarthritis”, “prevents and treats the degradation of articular cartilage”, “promotes and maintains joint health”, or an equivalent expression printed on the package. Any package configuration and packaging material suitable for containing the composition is useful in the invention, e.g., bag, box, bottle, can, pouch, and the like manufactured from paper, plastic, foil, metal, and the like. In a preferred embodiment, the package further comprises the composition of the invention. In a preferred embodiment, the package contains a food composition adapted for a particular animal such as a human, canine, or feline, as appropriate for the label, preferably a companion animal food composition for dogs or cats. In a preferred embodiment, the package is a can or pouch comprising a food composition of the invention. In various embodiments, the package further comprises at least one window that permit the package contents to be viewed without opening the package. In some embodiments, the window is a transparent portion of the packaging material. In others, the window is a missing portion of the packaging material.

In another aspect, the invention provides for use of DGLA to prepare a medicament for one or more of preventing and treating osteoarthritis; preventing and treating the degradation of articular cartilage; promoting and maintaining joint health; improving the quality of life; and promoting the health and wellness in an animal. Generally, medicaments are prepared by admixing a compound or composition, i.e., DGLA or a DGLA composition, with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

### EXAMPLES

The invention can be further illustrated by the following example, although it will be understood that this example is included merely for purposes of illustration and is not intended to limit the scope of the invention unless otherwise specifically indicated.

### Example 1

Canine cartilage was digested in a 37° C. shaking water bath using the following enzymes: trypsin (0.25%) for 25 minutes, hyaluronidase (150U/ml) for 1 hour, and collagenase (0.78%) overnight. Digested cartilage was filtered to obtain chondrocytes. Chondrocytes were expanded in monolayer culture with complete media. Composition of the complete media is Dulbecco's Modified Eagle Medium (DMEM)/F12 supplemented with 10% FBS, 1% P/S (100 U/mL penicillin and 100 μg/mL streptomycin) and 50 μg/ml ascorbic acid. Reaching subconfluence, chondrocytes were detached with 0.05% trypsin and cryopreserved. After cryopreservation, canine chondrocytes were expanded in a monolayer and, after reaching subconfluence, cells were trypsinized and subsequently immobilized in alginate beads. Cells suspended in complete media +3% alginate were dropped from a 18-gauge syringe into calcium chloride (120 mM) to form “beads.” Chondrocyte beads were cultured in complete media and media was changed every 2 or 3 days. At the end of the treatment, the chondrocyte beads were dissolved in sodium citrate (55 mM in 0.15 M NaCl). Suspensions were centrifuged at 2620 rpm for 5 minutes. Cells were washed with phosphate buffer and centrifuged again at 2620 rpm for 5 minutes. One mL RNAqueous™ lysis binding solution (Ambion, Austin, Tex.) was added to the isolated canine chondrocyte pellet, mixed thoroughly and stored at −80° C. until RNA isolation could be performed.

Samples were vortexed and homogenized using a Quiashredder column (Qiagen, Valencia, Calif.) according to manufacturer's directions. The homogenized lysate was collected and 1 equal volume of 64% ethanol was added to it. This mixture was then applied to an RNAqueous™ filter cartridge, 700 uL at a time, and centrifuged for 1 minute at 10,000 rpm. The cartridge was washed using 700 uL wash solution #1 and 500 uL wash solution #2/3 with centrifugation at 10,000 rpm for 1 minute for each wash. The filter cartridge was dried by centrifugation (10,000 rpm) for 1 minute. RNA was eluted 3 times by centrifugation (as above) using 30 uL aliquots of 95-100° C. elution solution. The resulting RNA was DNAse-treated and quantitated in a Beckman DU 640B spectrophotometer (Beckman Coulter, Inc., Brea, Calif.) at 260 nm. Additionally, quantity and quality were assessed using a bioanalyzer (Agilent, Santa Clara, Calif.) according to manufacturer's directions.

Stimulated monocyte neutrophil conditioned media (SMNCM) was made by isolating monocytes and neutrophils from canine whole blood using NycoPrep™ according to the manufacturer's directions. Monocytes and neutrophils were stimulated with lipopolysaccharide (20 ng/mL) for 72 hours. The resulting supernatant was used as SMNCM in cell culture experimentation (SMNCM made up 10% of media used during experimentation and is kept in frozen aliquots).

Chondrocytes were enriched with a fatty acid at 50 μM just after they were enclosed in beads. FBS and ethanol were used as a vehicle. Once the enriched media were prepared, ethanol represented less than 0.1%. Cells were enriched with either arachidonic acid (AA) and dihomo-γ-linolenic acid for 3 weeks total in complete media. The 3^{rd} week the enriched media also contained 10% stimulated monocyte neutrophil conditioned media (SMNCM). Chondrocyte beads were dissolved at the end of the experiment.

All samples were run singularly against each primer/probe set to determine what standard curve should be used. Standard curves were generated using serial dilutions of liver RNA or RNA from experimental samples. Alternately, if the samples did not fall within either of the curve ranges, the sample with the lowest C_{T} (cycle threshold) would be re-reverse transcribed and a 1:10 serial dilution would be used as the standard curve for that primer/probe set. Values were normalized to cyclophilin A levels as determined by quantitative PCR. Inductions were calculated from each of the lowest sample's normalized value. N=4 for all treatments.

TABLE 1
Quantitative PCR Expression Values

| Gene | AA | DGLA |
|---|---|---|
| IL-6 | 4.8 | 3.9 |
| 2252B | 4.3 | 4.7 |
| MMP-9 | 9.5 | 22.9 |
| IGF-2 | 14.7 | 15.9 |
| TNF-alpha | 26.4 | 18.8 |
| ADAMTS-5 | 3.0 | 2.8 |
| GRIA-4 | 5.0 | 4.2 |
| TIMP-1 | 3.5 | 3.7 |
| IL-1 | 4.1 | 2.4 |
| IL-8 | 3.0 | 3.1 |
| MMP-2 | 1.8 | 1.6 |
| MMP-13 | 2.7 | 2.1 |
| PC-2A | 4.5 | 5.2 |
| ADAMTS-4 | 1.4 | 1.6 |
| CYTL-1 | 18.1 | 18.0 |
| SOX-9 | 1.9 | 1.9 |
| TIMP-2 | 1.6 | 1.6 |

Values represent relative average induction values. Abbreviations: Interleukin (IL); Matrix Metalloproteinase (MMP); Insulin-like Growth Factor (IGF); Tumor Necrosis Factor (TNF); Procollagen (PC); Cytokine-like (CYTE); SRY (sex determining region Y)-box (SOX); Cytokine-like (CYTL); Glutamate Receptor, Ionotrophic, AMPA (GRIA); SRY (sex determining region Y)-box (SOX); A Disintegin-like and Metallopeptidase with Thrombospondin Type 1 Motif (ADAMTS); Tissue Inhibitor of Metalloproteinase (TIMP).

### Example 2

HT-29 clone 34 cells were seeded at 2.0×10⁵ cells/well in DMEM with 5% FCS and 1% PS. Cells were incubated for 24 hours at 37° C. at 5% CO₂ with or without rhTNF-alpha with dihomo-gamma-linolenic acid (0.1, 0.3, 0.5, 0.7 or 1 mg/ml) in ethanol as a vehicle. NF-kB activity was measured as SEAP release using a Phospha-Light™ System (Applied Biosystems, Bedford, Mass.) per manufacturer's directions.

TABLE 2
NF-kB Activation
NF-kB Activation

| | DGLA mg/ml | | | | |
|---|---|---|---|---|---|
| Treatment | 0.1 | 0.3 | 0.5 | 0.7 | 1.0 |
| −TNF-alpha (mean) | 10 | 12 | 10 | 7 | 7 |
| +TNF-alpha (mean) | 362 | 338 | 313 | 289 | 210 |
| Inhibition (mean) | 16 | 22 | 28 | 33 | 51 |

### Example 3

Cell media was aliquoted in micro-centrifuge tubes and stored at −80° C. until analysis. Cells were enriched with either arachidonic acid (AA) and dihomo-γ-linolenic acid. Prostaglandin E2 (PGE₂) levels were determined using Amersham Prostaglandin Prostaglandin E₂ Biotrak Enzyme Immunoassay (EIA, GE Healthcare). Before performing the assay, media samples were thawed then diluted 50 to 250 times in assay buffer. Standards and samples were run in duplicate.

The cell suspensions obtained after dissolving the alginate beads were centrifuged. After removing the supernatant, each cell pellet was resuspended in 1 ml of phosphate buffered saline. From each sample, 10 μL was added to the same volume of trypan blue stain before counting with a hemocytometer.

TABLE 3
PGE2 Inhibition
PGE2 levels from chondrocyte media (fg/ml/cell)

| | Chondrocyte Batch: | | | | |
|---|---|---|---|---|---|
| | 31010 | 1084 | 31510 | 41210 | Avg |
| 50 μM AA | 473 | 957 | 274 | 3950 | 1414 |
| 50 μM DGLA | 331 | 800 | 182 | 1080 | 598 |
| % Decrease | 30 | 16 | 34 | 73 | 58 |

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A method for preventing and treating the degradation of articular cartilage in an animal comprising administering to the animal a therapeutically effective amount of DGLA.

13. The method of claim 12 wherein DGLA is administered in amounts of from about 0.01 to about 100 mg/kg/day.

14. The method of claim 12 wherein DGLA is administered to the animal on a regular basis.

15. The method of claim 12 wherein the animal is suffering from the degradation of articular cartilage.

16. The method of claim 12 wherein DGLA is administered as a dietary supplement or a food composition.

17. The method of claim 16 wherein the composition is a food composition and DGLA comprises from about 0.001 to about 40% of the food composition.

18. The method of claim 16 wherein the composition or dietary supplement further comprises one or more probiotics; inactivated probiotics; components of inactivated probiotics that promote health benefits similar to or the same as the probiotics; and one or more prebiotics.

19. The method of claim 12 wherein the animal is a human or a companion animal.

20. The method of claim 19 wherein the companion animal is a canine.

21. The method of claim 19 wherein the companion animal is a feline.

22. The method of claim 12 wherein the animal is an aging animal.

23. A method for promoting and maintaining joint health in an animal comprising administering to the animal a therapeutically effective amount of DGLA.

24. The method of claim 23 wherein DGLA is administered in amounts of from about 0.01 to about 100 mg/kg/day.

25. The method of claim 23 wherein DGLA is administered to the animal on a regular basis.

26. The method of claim 23 wherein DGLA is administered as a dietary supplement or a food composition.

27. The method of claim 26 wherein the composition is a food composition and DGLA comprises from about 0.001 to about 40% of the food composition.

28. The method of claim 26 wherein the composition or dietary supplement further comprises one or more probiotics; inactivated probiotics; components of inactivated probiotics that promote health benefits similar to or the same as the probiotics; and one or more prebiotics.

29. The method of claim 23 wherein the animal is a human or a companion animal.

30. The method of claim 29 wherein the companion animal is a canine.

31. The method of claim 29 wherein the companion animal is a feline.

32. The method of claim 23 wherein the animal is an aging animal.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51 (canceled)

52. (canceled)

53. A composition comprising DGLA in a therapeutically effective amount for one or more of preventing and treating osteoarthritis, preventing and treating the degradation of articular cartilage, and promoting and maintaining joint health.

54. The composition of claim 53 containing DGLA in amounts sufficient to administer DGLA to an animal in amounts to about from about 0.01 to about 100 mg/kg/day.

55. A pharmaceutical or nutraceutical composition comprising DGLA and one or more pharmaceutically or nutraceutically acceptable carrier, diluents or excipients.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)
